(19) United States
(12) Patent Application Publication
(10) Pub. No.: US 2010/0186752 A1
(43) Pub. Date: Jul. 29, 2010

(54) DISPOSABLE CUFF LINER FOR USE WITH A BLOOD PRESSURE CUFF

(76) Inventor: Sandy Rixson, Yorba Linda, CA (US)

Correspondence Address: FLIESLER MEYER LLP, 650 CALIFORNIA STREET, 14TH FLOOR, SAN FRANCISCO, CA, 94108, US

(21) Appl. No.: 12/361,421

(22) Filed: Jan. 28, 2009

(51) Int. Cl. A61F 13/00

(52) U.S. Cl. 128/846

(57) ABSTRACT

A disposable cuff liner for use with a blood pressure cuff and means for using thereof. In accordance with an embodiment, the cuff liner can be easily wrapped around a patient's limb, and generally approximates the width of a typical blood pressure cuff. The cuff liner can be shaped to better conform to the shape of the limb. An adhesive strip allows the cuff liner to be wrapped around the limb and then fixed into place. Sizing indicators can be marked on the cuff liner to allow for sizing and more accurate positioning of the two ends of the cuff liner. In accordance with an embodiment, the sizing indicators can be perforated, to allow the cuff liner to be easily customized for size. Once applied to the patient, the blood pressure cuff of a sphygmomanometer can be wrapped around the patient's limb and over the cuff liner, to measure the patient's blood pressure.

### FIELD OF INVENTION

The invention is generally related to medical equipment, medical supplies, and blood pressure monitoring equipment, and is particularly related to a disposable cuff liner for use with a blood pressure cuff and means for using thereof.

### BACKGROUND

Throughout the world, and particularly within the United States, healthcare has become an increasingly important aspect of everyday life. With advances in medical procedures and pharmaceuticals, a person's life expectancy and their expectation as to overall quality of life has steadily improved with each generation. Regular checkup visits to a family physician are common, and the frequency of both required and/or elective surgeries is ever increasing.

With this increase in medical care comes the increased risk of contracting illnesses in medical environments, particularly in hospitals. While all medical facilities maintain scrupulous hygiene, there remains the possibility of contracting a wide range of infections from common cold, or skin rash to more serious illnesses such as pneumonia, or severe acute respiratory syndrome (SARS). Many of these ailments can be contracted through inadvertent passing of germs and bacteria via common surfaces, which then leads to cross-infection of other patients and medical practitioners. The potential for contracting an illness is particularly acute for those patients whose immune systems may already be weakened, or who are undergoing surgery. Such infections are often referred to as nosocomial infections, or hospital acquired infections (HAI).

A particular problem exists with the measuring of blood pressure, which is performed using a sphygmomanometer or blood pressure device. This type of device generally includes a blood pressure cuff, which wrapped around a patient's limb (such as their arm or leg), and inflated to measure the patient's blood pressure. Measurement of blood pressure is an extremely common diagnostic procedure, and may be performed at a physician's office or hospital during each patient checkup. Anesthesiologists may continuously measure a patient's blood pressure for several hours during surgical procedures. However, since the blood pressure cuff is commonly used for every patient it potentially acts as a fomite, carrying infectious organisms such as bacteria, and then transferring those infectious organisms from one individual to another. Techniques that can be used to reduce these problems are of great value to the healthcare industry.

### SUMMARY

Described herein is a disposable cuff liner for use with a blood pressure cuff and means for using thereof. In accordance with an embodiment, the cuff liner can be easily wrapped around a patient's limb, and generally approximates the width of a typical blood pressure cuff. The cuff liner can be shaped to better conform to the shape of the limb. An adhesive strip allows the cuff liner to be wrapped around the limb and then fixed into place. Sizing indicators can be marked on the cuff liner to allow for sizing, and for more accurate positioning of the two ends of the cuff liner. In accordance with an embodiment, the sizing indicators can be perforated, to allow the cuff liner to be easily customized for size by tearing, either before or after placement on the patient. Once applied to the patient, the blood pressure cuff of a sphygmomanometer can be wrapped around the patient's limb and over the cuff liner, to measure the patient's blood pressure. In this manner the blood pressure cuff is generally prevented from coming in direct contact with the patient's skin. This helps both to prevent contamination of the cuff, and minimize irritation of the patient's skin. Once the patient's blood pressure has been measured, the cuff liner can disposed, and a new cuff liner used for each new patient.

### BRIEF DESCRIPTION OF THE DRAWINGS

FIG. 1 shows a disposable cuff liner for use with a blood pressure cuff, in accordance with an embodiment.

FIG. 2 shows different sizes of disposable cuff liners, in accordance with different embodiments.

FIG. 3 shows a disposable cuff liner as it may be used with a blood pressure cuff, in accordance with an embodiment.

FIG. 4 shows a disposable elasticated cuff liner for use with a blood pressure cuff, in accordance with an alternative embodiment.

FIG. 5 shows a disposable cuff liner as it may be adjusted and used with different patients, in accordance with an embodiment.

### DETAILED DESCRIPTION

Described herein is a disposable cuff liner for use with a blood pressure cuff and means for using thereof. In accordance with an embodiment, the cuff liner can be easily wrapped around a patient's limb, and generally approximates the width of a typical blood pressure cuff. The cuff liner can be shaped to better conform to the shape of the limb. An adhesive strip allows the cuff liner to be wrapped around the limb and then fixed into place. Sizing indicators can be marked on the cuff liner to allow for sizing, and for more accurate positioning of the two ends of the cuff liner. In accordance with an embodiment, the sizing indicators can be perforated, to allow the cuff liner to be easily customized for size by tearing, either before or after placement on the patient. Once applied to the patient, the blood pressure cuff of a sphygmomanometer can be wrapped around the patient's limb and over the cuff liner, to measure the patient's blood pressure. In this manner the blood pressure cuff is generally prevented from coming in direct contact with the patient's skin. This helps both to prevent contamination of the cuff, and minimize irritation of the patient's skin. Once the patient's blood pressure has been measured, the cuff liner can disposed, and a new cuff liner used for each new patient.

In accordance with an embodiment, the use of a disposable cuff liner can also help to reduce or substantially prevent skin erosion in a patient. Particularly, in surgical environments, an anesthesiologist may apply a blood pressure cuff to the patient for many (sometimes 6 or more) hours. During surgery, the patient's blood pressure is typically measured at cuff pressures of approximately 180-200 mmHg, at each 3-5 minute interval. When a blood pressure cuff is used for such a long period of time and regularity it can lead to localized problems, such as pinching of the patient's skin, which in turn can cause petechiae or minor hemorrhaging of the blood vessels under the skin. This is of particular concern with patients who have a fragile skin condition due to the nature of patient's illness, gender, age or preexisting systemic disease. Younger children and infants are also prone to damaging their fragile skin. In accordance with an embodiment, the disposable cuff liner can be made in different materials and sizes to best suit and protect the particular needs of the target patient.

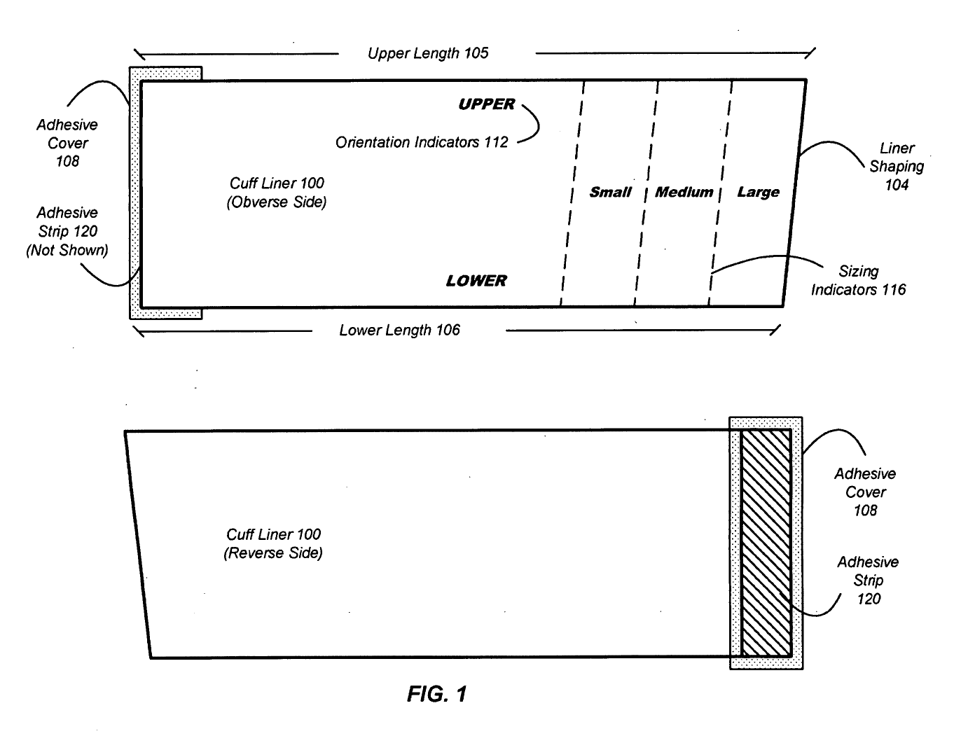

FIG. 1 shows a disposable cuff liner for use with a blood pressure cuff, in accordance with an embodiment. As shown in FIG. 1, the cuff liner 100 can be fabricated from any suitable cuff liner material. In accordance with an embodiment the cuff liner material can be a soft paper cloth or fabric, in either a single-ply or multi-ply or multi-layered construction, such as is commonly used to make disposable medical gowns, since this type of material is both thin enough that it does not prevent accurate blood pressure readings, and soft enough that it is unlikely to irritate the skin or cause discomfort to the patient. The paper cloth material is also generally resistant to wetting so it is less permeable to bacteria, which is a particular benefit in surgical environments.

The cuff liner is made with a long-enough longitudinal dimension that it can be easily wrapped around a patient's arm or leg, and of a wide-enough lateral dimension that it generally approximates the width of a typical blood pressure cuff. Since the circumference of a patient's limb is generally tapered from the torso outward, the cuff liner can be shaped 104 at a first longitudinal end to better conform to the tapered shape of the limb, and so that it's upper length 105 is somewhat longer that its lower length 106. Some illustrative examples of possible dimensions are provided below. Orientation indicators 112 assist the medical practitioner in properly orienting the placement of the cuff liner relative to the orientation of the limb. Although FIG. 1 shows textual indicators, in accordance with other embodiments pictorial indicators can be used instead. An adhesive strip 120 on the reverse side of the cuff liner and at the opposite longitudinal end allows the cuff liner to be wrapped around the limb and then fixed into place using the adhesive strip. Prior to use, the adhesive strip is covered with an adhesive cover 108 which can simply be removed by the medical practitioner and discarded to expose the adhesive. To better assist the medical practitioner in placing the cuff liner about the patient's limb, sizing indicators 116, such as “small” “medium” and “large” or some numeric equivalents, can be marked (shown in FIG. 1 by the dotted lines) on the obverse side of the cuff liner. This allows for sizing of the cuff liner, and for more accurate positioning of the two ends of the cuff liner once wrapped and adhered. In accordance with some embodiments, the sizing indicators can be perforated, to allow the cuff liner to be easily customized for size by tearing, either before or after placement on the patient.

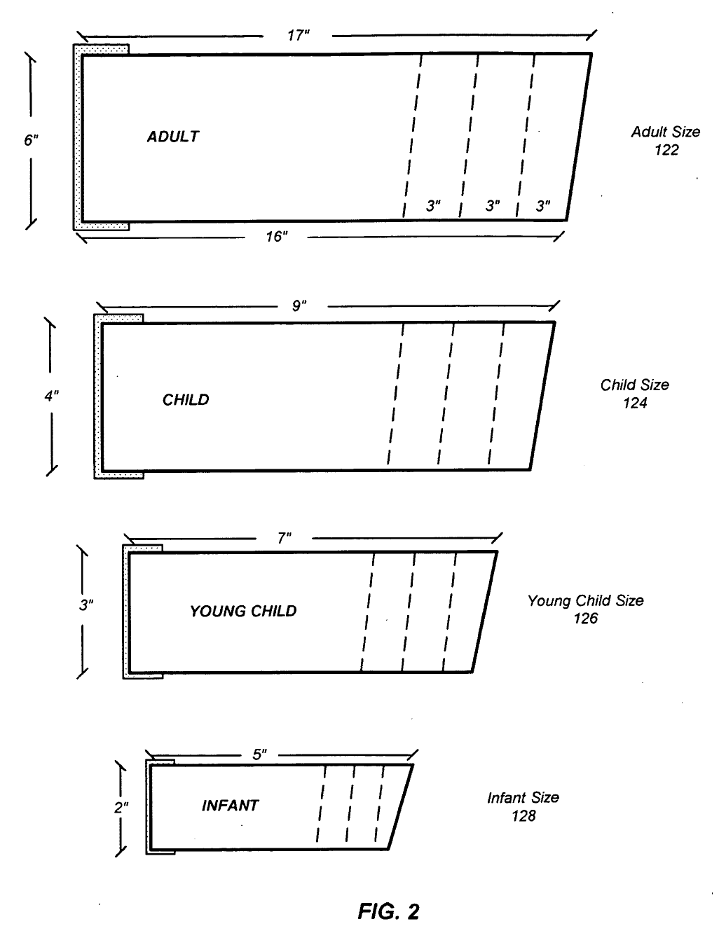

FIG. 2 shows different sizes of disposable cuff liners, in accordance with different embodiments. As described above, the protection of the patient's skin during surgical procedures is of particular concern with those patients who have fragile skin, including older adults, younger children, and infants. Furthermore, each of these types of patient often requires a different size of blood pressure cuff that would typically be used for that patient. In accordance with an embodiment, the disposable cuff liner can be made in different sizes to best suit the particular needs of the target patient. As shown in FIG. 2, an adult-sized cuff liner 122 can be made in approximate dimensions of, for example, 17″ long×6″ wide, to suit the size of a typical adult and a typical adult-sized blood pressure cuff. Such a cuff liner can have tear-off portion at 3″ intervals to allow for optimal sizing for an adult patient. The cuff liner can be proportionally scaled-down to suit different patient types, so that, for example a child-sized cuff liner 124 can be made in approximate dimensions of, for example, 9″ long×4″ wide, to suit the size of a typical child and a typical child-sized blood pressure cuff, a young-child-sized cuff liner 126 can be made in approximate dimensions of, for example, 7″ long×3″ wide, to suit the size of a typical young child and a typical young-child-sized blood pressure cuff, and an infant-sized cuff liner 128 can be made in approximate dimensions of, for example, 5″ long×2″ wide, to suit the size of a typical infant and a typical infant-sized blood pressure cuff. It will be evident that the sizes and dimensions shown are just some examples, and that different shapes and sizes may be developed within the spirit and scope of the invention for different patient needs.

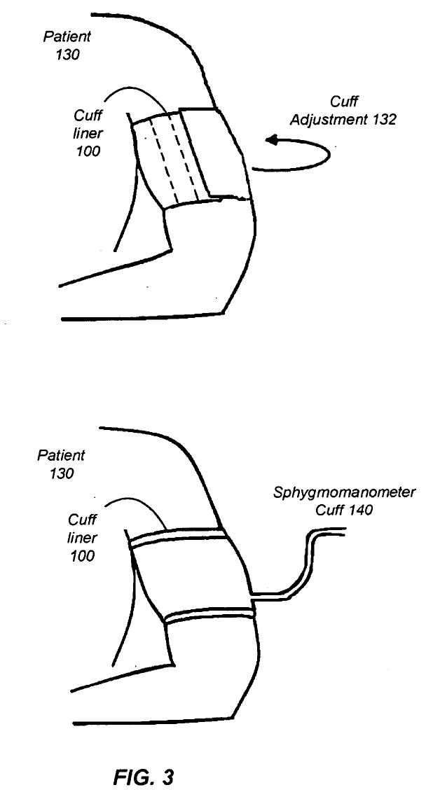

FIG. 3 shows a disposable cuff liner as it may be used with a blood pressure cuff, in accordance with an embodiment. As shown in FIG. 3, the cuff liner 100 can be placed on the limb of the patient 130, and adjusted 132 for optimal fit. FIG. 3 illustrates the cuff liner being used with the patient's arm; however the cuff liner can similarly be used with the patient's leg as appropriate. As described above, in accordance with an embodiment, the cuff liner can be shaped to better conform to the tapered shape of the limb. Orientation indicators assist the medical practitioner in properly orienting the placement of the cuff liner relative to the orientation of the limb, while sizing indicators allow for adjustment of size, and for more accurate positioning of the two ends of the cuff liner once wrapped and adhered. The blood pressure cuff of a sphygmomanometer 140 can then be wrapped around the patient's limb and over the cuff liner, and inflated to measure the patient's blood pressure. In this manner the blood pressure cuff is generally prevented from coming in direct contact with the patient's skin, which helps to prevent contamination of the cuff, and infection of the patient. Once the patient's blood pressure has been measured (or in the case of a surgery following the surgical and/or post-operative procedures), the cuff liner can be removed and disposed. A new cuff liner can be used for each new patient.

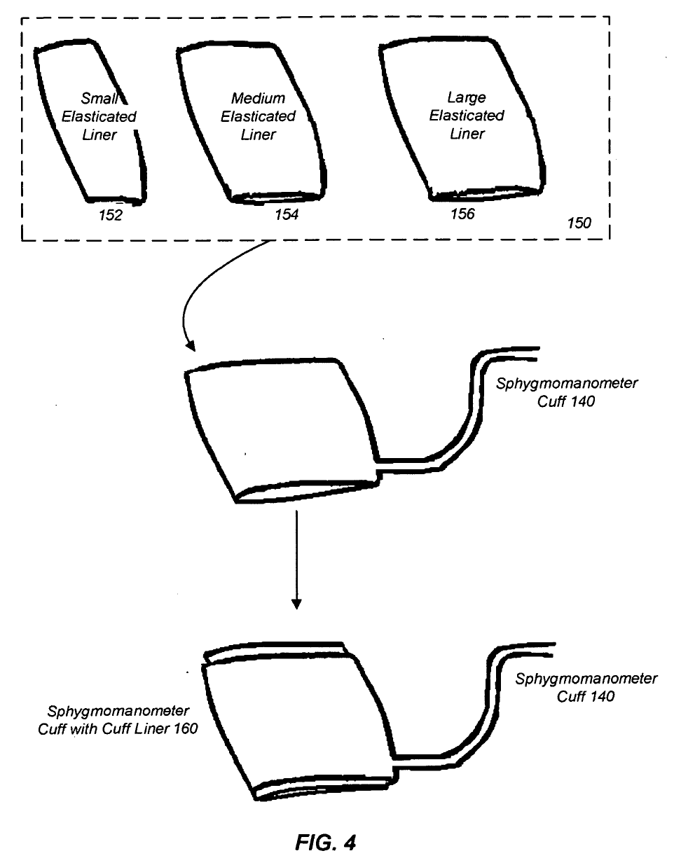

FIG. 4 shows a disposable elasticated cuff liner for use with a blood pressure cuff, in accordance with an alternative embodiment. As shown in FIG. 4, in an alternative embodiment the cuff liner 150 can be fabricated from an elasticated cuff liner material. In accordance with an embodiment this cuff liner material can be a soft flexible woven or non-woven cloth or fabric, such as is commonly used to make elasticated bandages, since this type of material is similarly thin enough that it does not prevent accurate blood pressure readings, and soft enough that it is unlikely to irritate the skin or cause discomfort to the patient. The woven or non-woven cloth material is generally not resistant to wetting, so it is more suited to non-surgical environments, such as general physician care and post-operative treatment. In the embodiment shown in FIG. 4, the cuff liner is cylindrical in shape, with a long enough circumference that it can be easily wrapped around a patient's arm or leg, and of a wide enough lateral dimension that it generally approximates the width of a typical blood pressure cuff. To better assist the medical practitioner in placing the cuff liner about the patient's limb, the cuff liner can be made in, e.g. “small” 152, “medium” 154, and “large” 156 or some other equivalents. As with the earlier embodiments, the medical practitioner can place the cuff liner around the limb; and the blood pressure cuff of a sphygmomanometer can then be wrapped around the patient's limb and over the cuff liner 160, and inflated to measure the patient's blood pressure. Again, the blood pressure cuff never comes in direct contact with the patient's skin, which helps to prevent contamination of the cuff. Once the patient's blood pressure has been measured, the cuff liner can be disposed, and a new cuff liner can be used for each new patient.

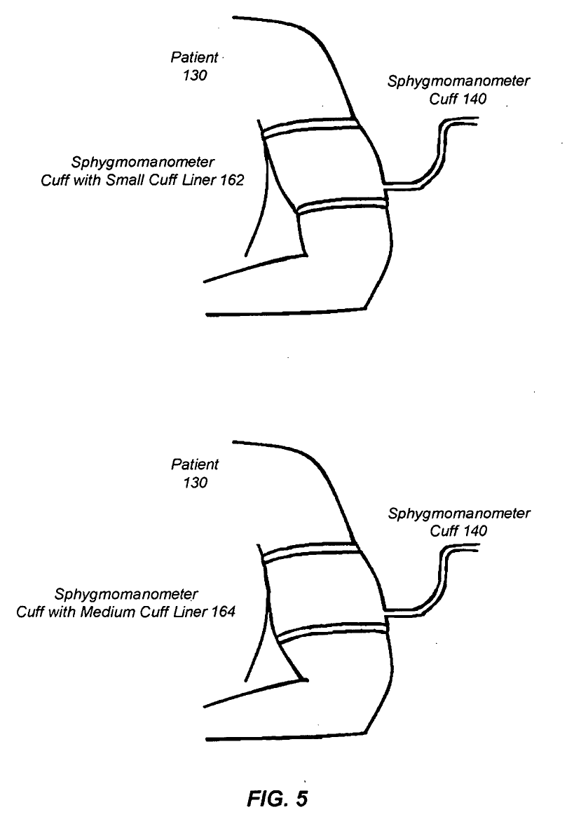

FIG. 5 shows a disposable cuff liner as it may be adjusted and used with different patients, in accordance with an embodiment. As shown in FIG. 5, regardless of which embodiment is employed, the cuff liner can be placed on the limb of the patient, and adjusted for optimal fit, including for a smaller patient 162 or a larger patient 164. While FIG. 5 illustrates a cuff liner being used with the patient's arm, it will be evident that these are labels, and that in some instances a smaller cuff liner might be suitable and labeled for “arm” use, and a larger cuff liner might be suitable and labeled for “leg” use.

The foregoing description of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention for various embodiments and with various modifications that are suited to the particular use contemplated. For example, the lengths and widths of the cuff liner can be altered to suit particular types of applications, or blood pressure cuffs; and different types of materials can be used to make the cuff liner. It is intended that the scope of the invention be defined by the following claims and their equivalence.

What is claimed is:

1. A disposable cuff liner for use with the blood pressure cuff of a sphygmomanometer, comprising:
a soft cloth fabric cuff liner with an obverse side and a reverse side, a longitudinal dimension such that it can be wrapped around a patient's limb, and a lateral dimension that it generally approximates the width of a typical blood pressure cuff;
an adhesive strip on the reverse side of the cuff liner at a first longitudinal end, that allows the cuff liner to be wrapped around the limb and then fixed into place using the adhesive strip, wherein the adhesive strip is initially covered with an adhesive cover which can be removed by a medical practitioner and discarded to expose the adhesive; and
wherein the cuff liner can be applied to the limb and affixed into place, and the blood pressure cuff of a sphygmomanometer then wrapped around the limb and over the cuff liner, and used to measure the patient's blood pressure so that the blood pressure cuff is generally prevented from direct contact with the patient's skin, and wherein once the patient's blood pressure has been measured, the cuff liner can be removed and disposed.

2. The disposable cuff liner of claim 1, wherein the cuff liner is made of a wetting-resistant paper.

3. The disposable cuff liner of claim 1, wherein the cuff liner is shaped at the other longitudinal end to better conform to the tapered shape of the patient's limb.

4. The disposable cuff liner of claim 1, further comprising orientation indicators printed on an obverse side of the cuff liner to assist a medical practitioner in properly orienting the placement of the cuff liner relative to the orientation of the patient's limb.

5. The disposable cuff liner of claim 1, further comprising sizing indicators such as “small” “medium” and “large” or numeric equivalents, marked on the obverse side of the cuff liner to allow for sizing, and more accurate positioning of the two ends of the cuff liner once wrapped and adhered.

6. The disposable cuff liner of claim 4, wherein the sizing indicators are perforated, to allow the cuff liner to be easily customized for size by tearing, either before or after placement on the patient.

7. A disposable cuff liner for use with the blood pressure cuff of a sphygmomanometer, comprising:
a soft wetting-resistant paper cloth fabric cuff liner with an obverse side and a reverse side, a longitudinal dimension such that it can be wrapped around a patient's limb, and a lateral dimension that it generally approximates the width of a typical blood pressure cuff, and wherein the fabric is shaped at a first longitudinal end to better conform to the tapered shape of the patient's limb;
an adhesive strip on the reverse side of the cuff liner at the other longitudinal end, that allows the cuff liner to be wrapped around the limb and then fixed into place using the adhesive strip, wherein the adhesive strip is initially covered with an adhesive cover which can be removed by a medical practitioner and discarded to expose the adhesive;
orientation indicators printed on an obverse side of the cuff liner to assist a medical practitioner in properly orienting the placement of the cuff liner relative to the orientation of the patient's arm or leg; sizing indicators, marked on the obverse side of the cuff liner and perforated to allow for sizing either before or after placement on the patient, and more accurate positioning of the two ends of the cuff liner once wrapped and adhered; and
wherein the cuff liner can be applied to the limb and affixed into place, and the blood pressure cuff of a sphygmomanometer then wrapped around the limb and over the cuff liner, and used to measure the patient's blood pressure so that the blood pressure cuff is generally prevented from direct contact with the patient's skin, and wherein once the patient's blood pressure has been measured, the cuff liner can be removed and disposed.

8. A disposable cuff liner for use with the blood pressure cuff of a sphygmomanometer, comprising:
a plurality of different sized elasticated fabric form cuff liners, each shaped in the form of a cylinder and having a circumference such that it can be wrapped around a patient's limb, and a lateral dimension that generally approximates the width of a typical blood pressure cuff;
wherein an appropriately-sized cuff liner can be selected and applied to the limb, and the blood pressure cuff of a sphygmomanometer then wrapped around the limb and over the cuff liner, and used to measure the patient's blood pressure so that the blood pressure cuff is generally prevented from direct contact with the patient's skin, and wherein once the patient's blood pressure has been measured, the cuff liner can be removed and disposed.
